(19) United States
(12) Plant Patent Application Publication
(10) Pub. No.: US 2003/0150037 P1
(43) Pub. Date: Aug. 7, 2003

(54) VERBENA PLANT NAMED 'SUNMARICOS'

(76) Inventor: Yasunori Yomo, Yasu-gun (JP)

Correspondence Address: BURNS, DOANE, SWECKER & MATHIS L.L.P., P.O. Box 1404, Alexandria, VA, 22313-1404, US

(21) Appl. No.: 10/066,655

(22) Filed: Feb. 6, 2002

(51) Int. Cl. A01H 5/00

(52) U.S. Cl. PLT/308

(57) ABSTRACT

Disclosed herein is a verbena plant, which has a broad, spreading growth habit and long stems. The plant forms flowers in clusters with a great profusion of blooms. The blooming period is from late April to November and the flowering duration is long. The entire plant remains in bloom for an extended period of time. The flower size is large and the petal color of the flowers is deep purplish pink. The plant is highly tolerant to heat, and exhibits a high resistance to pests and diseases, particularly powdery mildew, and has a high resistance to rain.

### BACKGROUND OF THE VARIETY

The present invention provides a new and distinct variety of Verbena×hybrida plant obtained from open-pollination with variety ‘114-7’ (Verbena×hybrida) and an unknown verbena plant. Variety ‘114-7’ was developed from crossing a Verbena×hybrida named ‘Amethyst’ (non-patented in the United States) (female) and a wild type of verbena plant (Verbena peruviana) native to Brazil.

The verbena is a very popular plant and is used for flower bedding and potting in the summer season. There are only a few varieties of Verbena plants which have a spreading growth habit, much branching, a large number of flowers in a cluster and which have a high resistance to rain, heat, cold and diseases. Accordingly, this invention is aimed at obtaining a new variety having a spreading growth habit, strong branching, a large number of flowers in a cluster, a flower of large diameter, a high tolerance to heat and cold, and resistance to diseases and pests, and also having petals that are deep purplish pink.

First of all, variety ‘114-7’ was developed from crossing a Verbena×hybrida named ‘Amethyst’ (not patented in the United States) (female) and a wild type of verbena plant (Verbena peruviana) native to Brazil (male). In August 1997, seedlings resulting from open-pollination with variety ‘114-7’ (Verbena×hybrida) and an unknown verbena plant were obtained at the Hakushu Nursery Center of SUNTORY Ltd., located at 2913-1, Torihara, Hakushu-cho, Kitakoma-gun, Yamanashi-ken, Japan. In January 1998, a seedling was selected in view of its spreading growth habit, content of less bluish and more reddish pigment in flower than its parent and having deep purplish pink petals. Then, it was propagated by cuttings, and was grown as a trial in flowerbeds and planters beginning in April 1998 in the Hakushu Nursery Center of SUNTORY Ltd., at 2913-1, Torihara, Hakushu-cho, Kitakoma-gun, Yamanashi-ken, Japan, and the Omi Research Center of SUNTORY Ltd., located at 863-1, Aza-Iketani, Omori-cho, Yokaichi-shi, Shiga-ken, Japan. The botanical characteristics of the plant were examined using a similar variety ‘Sunmaripi’ and ‘Sunmarisa’ for comparison. As a result, it was concluded that this discovered verbena plant is distinguishable from other varieties whose existence is known, uniform and stable in its characteristics. Then, this new variety of verbena plant was named ‘Sunmaricos’.

In the following description, the color-coding is in accordance with the Horticultural Color Chart of The Royal Horticultural Society, London, England (R.H.S. Color Chart), and the Inter-Society color Council-Nation Bureau of Standard Color Name (I.S.C.C.-N.B.S. Color Name). A color chart based on The Japan Color Standard for Horticultural Plant (J.H.S. Color Chart) is also added for reference.

The parent variety ‘114-7’ used in obtaining this new variety ‘Sunmaricos’ was developed from crossing a Verbena×hybrida named ‘Amethyst’ (not patented in the United States) (female) and a wild type of verbena plant (Verbena peruviana) native to Brazil (male), is presently maintained at the Ohmi R&D Center of Suntory Ltd. The main botanical characteristics of the parent variety ‘114-7’ are as follows, as grown at the Ohmi R&D Center of Suntory Ltd.

Plant:

Growth habit.—Spreading.

Plant height.—15-20 cm.

Plant extension.—60-100 cm.

Stem:

Diameter.—1.5-2.5 mm.

Anthocyanin pigmentation.—Present.

Branching.—Abundant.

Pubescence.—Medium.

Length of internodes.—2.5-4.5 cm.

Leaf:

Phyllotaxis.—Opposite.

Shape of blade.—Hastate.

Length.—3.0-4.5 cm.

Width.—2.0-2.5 cm.

Depth of incision.—Shallow.

Color.—Dark olive green (R.H.S. 137B, J.H.S. 3707).

Pubescence.—Sparse.

Flower:

Facing direction.—Upward.

Outward curvature of petal.—Flat.

Diameter.—1.5-2.0 cm.

Height.—2.0-2.5 cm.

Color.—Vivid purple (R.H.S. 88A, J.H.S. 8606).

Color presentation.—Uneven.

Overlapping of petals.—Separated.

Shape of cluster.—Obconical.

Length of cluster.—3.0-5.0 cm.

Diameter of cluster.—4.0-5.5 cm.

Calyx.—1.2-1.4 cm in length.

Anthocyanin pigmentation of calyx limb.—Present.

Peduncle.—1.0-2.0 mm in thickness; and 2.0-4.5 cm in length.

Number of flowers.—Many (approximately 11-14).

Reproductive organs.—1 pistil and 4 stamens.

Flower fragrance.—Absent.

Flowering duration.—Long.

Physiological and ecological characteristics: High resistance to diseases and pests and high tolerance to heat and cold.

The ‘Sunmaripi’ variety used as a comparison for this new ‘Sunmariro variety is one of our bred varieties and was filed in Japan [‘SUNMARIPI’ (Registration No. 4900 under the Seed and Seedlings Law of Japan)] and in the US [Verbena Plant Named ‘SUNVP-PI’ (Registration No. PLANT 10,705 as a plant patent)], and so on. The main botanical characteristics of the ‘Sunmaripi’ variety are as follows, as grown at the Ohmi R&D Center of Suntory Ltd.

Plant:

Growth habit.—Spreading.

Plant height.—15-25 cm.

Plant extension.—50-70 cm.

Growth.—Very vigorous with abundant branching and great profusion of blooms; the whole bush remaining in bloom for a considerable period of time.

Stem:

Diameter.—2.0-3.0 mm.

Anthocyanin pigmentation.—Present.

Branching.—Abundant.

Pubescence.—Medium.

Length of internode.—4.0-5.0 cm.

Leaf:

Phyllotaxis.—Opposite.

Shape of blade.—Hastate.

Length.—4.0-5.0 cm.

Width.—2.0-2.5 cm.

Depth of incision.—Shallow.

Color.—Grayish olive green (R.H.S. 137A-137B, J.H.S. 3716).

Pubescence.—Sparse.

Flower:

Facing direction.—Upward.

Outward curvature of petal.—Curved.

Diameter.—1.5-2.0 cm.

Height.—20 mm.

Color.—Deep purplish pink (R.H.S. 70C, J.H.S. 9213).

Color intensity.—Present.

Overlapping of petals.—Separate.

Spike.—30-35 mm in length; and 50-55 mm in diameter.

Calyx.—1.0 cm in length.

Anthocyanin pigmentation of calyx limb.—Present.

Peduncle.—2-3 mm in thickness; and 5.0-6.0 cm in length.

Number of flowers.—Plentiful (13-15).

Reproductive organs.—1 pistil and 5 stamens.

Flower fragrance.—Absent.

Flowering duration.—Long.

Physiological and ecological characteristics: High resistance to diseases and pests, particularly powdery mildew. High tolerance to heat and moderate tolerance to cold.

‘Sunmarisa’ used as a comparison for this new variety ‘Sunmaricos’ is one of our bred varieties and was filed in Japan [‘SUNMARIPI’ (Registration No. 8144 under the Seed and Seedlings Law of Japan)] and in the US [Verbena Plant Named ‘SUNVP-PI’ (Registration No. PLANT 11,130 as a plant patent)], and so on. The main botanical characteristics of ‘Sunmarisa’ are as follows, as grown at the Ohmi R&D Center of Suntory Ltd.

Growth habit.—Spreading.

Plant width.—65-71 cm.

Plant height.—Approximately 8-9 cm.

Stem:

Diameter.—1.7-2.2 mm.

Anthocyanin pigmentation.—Absent.

Pubescence.—Medium.

Branching.—Medium.

Length of internode.—Medium; 1.7-2.7 cm.

Leaf:

Phyllotaxis.—Opposite.

Shape of blade.—Oblong-lanceolate.

Depth of blade incision.—Shallow.

Length.—Medium; 2.9-3.6 cm.

Width.—Medium; 1.6-2.4 cm.

Color.—Dark olive green (R.H.S. 146A, J.H.S. 3707).

Pubescence.—Slight.

Petiole.—Present.

Diameter of petiole.—1.0-1.2 mm.

Length of petiole.—1.2-2.5 mm.

Flower:

Shape of cluster.—Obovate.

Length of cluster.—23-44 mm.

Diameter of cluster.—40-58 mm.

Facing direction.—Upward.

Outward curvature of petal.—Slightly curved.

Diameter.—1.8-1.9 cm.

Height.—2.1-2.2 cm.

Color.—Light purplish pink (R.H.S. 62C, J.H.S. 9503).

Eye color.—Pale yellow (R.H.S. 11D, J.H.S. 2503).

Eye size.—Small.

Color presentation.—Substantially even.

Overlapping of petals.—Separate.

Incision of petal.—Present.

Number of petals.—5.

Sepals.—Tubular in configuration.

Length of calyx.—1.2-1.4 cm.

Anthocyanin pigmentation of calyx limb.—Absent.

Color of anther.—Yellow green.

Diameter of peduncle.—0.9-1.4 mm.

Length of peduncle.—19-45 mm.

Color of peduncle.—Strong yellow green (R.H.S. 144A, J.H.S. 3507).

Number of flowers.—Commonly 13±2.

Flower bearing.—In a cluster.

Reproductive organs.—1 pistil and 4 stamens.

Flower fragrance.—Absent.

Flowering time.—Early.

Flowering duration.—Long.

Pollen.—Brilliant greenish-yellow (R.H.S. 6C, J.H.S. 2704) in coloration.

Physiological and ecological characteristics: High resistance to diseases and pests, particularly powdery mildew. High tolerance to heat and drought, high tolerance to rain, and medium tolerance to cold.

This new variety of verbena plant ‘Sunmaricos’ was asexually reproduced by cuttings at the Ohmi R&D Center of Suntory Ltd. The homogeneity and stability thereof were confirmed. The characteristics of the new variety are fully stable following such asexual reproduction.

### SUMMARY OF THE VARIETY

This new variety of verbena plant has a broad spreading growth habit and long stems. The plant is well branched and forms abundant flowers in a cluster. The blooms are present in profusion. The flowering time is early and blooming period is from early April to November and the flowering duration is long. The entire plant remains in bloom for an extended period of time. The flower size is large and the petal coloration of the flowers is vivid red. The plant is highly tolerant to heat, exhibits a high resistance to pests and diseases, particularly powdery mildew, and has a high resistance to rain.

### BRIEF DESCRIPTION OF THE DRAWINGS

FIG. 1 is a photograph of flowers of the new variety of verbena plant planted in the ground.

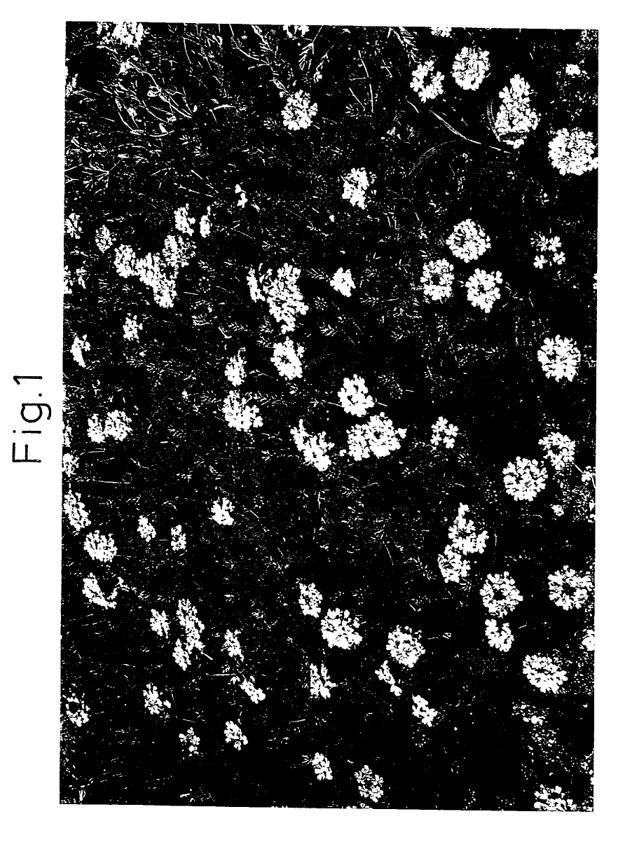

FIG. 2 is a photograph of flowers of the new variety of verbena plant.

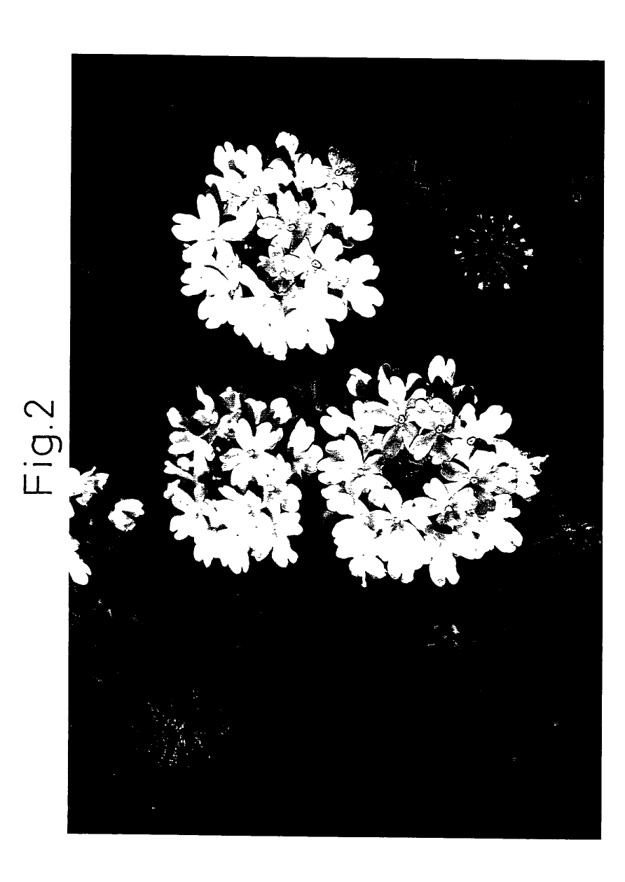

### DESCRIPTION OF THE VARIETY

The botanical characteristics of the new and distinct variety of verbena plant ‘Sunmaricos’ are as follows, as grown at the Ohmi R&D Center of Suntory Ltd.

Plant:

Growth habit.—Spreading.

Plant height.—12-15 cm.

Plant extension.—100-120 cm.

Stem:

Diameter.—2.0-2.5 mm.

Anthocyanin pigmentation.—Present.

Branching.—Abundant. Each single branch commonly produces two additional branches. More specifically, a single branch commonly becomes three branches and those three branches commonly produce nine branches, etc.

Pubescence.—Medium.

Subterranean stem.—Absent. However, when the stems contact the surface of the soil, the nodes take root in the ground and the plant growth thereby spreads.

Length of internodes.—2.5-4.5 cm.

Leaf:

Phyllotaxis.—Opposite.

Shape of blade.—Hastate.

Blade margin.—Serrate.

Length.—2.9-3.5 cm.

Width.—1.6-2.2 cm.

Depth of incision.—Absent.

Leaf apex.—Mucronate.

Leaf base.—Petiolate.

Color.—Dark olive green (R.H.S. 137B, J.H.S. 3707) on the upper surface and moderate olive green (R.H.S. 146A and J.H.S. 3508) on the under surface.

Pubescence.—Medium.

Petiole.—Present.

Petiole color.—Moderate olive green (R.H.S. 146A and J.H.S. 3508).

Diameter of petiole.—1.0-1.2 mm.

Length of petiole.—1.5-2.0 mm.

Flower:

Shape of cluster.—Obconical.

Length of cluster.—25-35 mm.

Diameter of cluster.—50-60 mm.

Facing direction.—Upward.

Outward curvature of petal.—Flat.

Diameter.—1.8-2.2 cm.

Height.—1.9-2.1 cm.

Color.—Deep purplish pink (R.H.S. 73A, J.H.S. 9205) on the upper surface and strong purple (R.H.S. 86B, J.H.S. 8310) on the under surface.

Color presentation.—variegations present.

Overlapping of petals.—Separate.

Length of Calyx.—1.1-1.2 cm.

Incision of petal.—Present.

Number of petals.—5.

Sepals.—Tubular in configuration.

Anthocyanin pigmentation of calyx limb.—Present.

Color of anther.—Pale yellow green. (R.H.S. 1D, J.H.S.3102).

Diameter of peduncle.—1.6-2.0 mm.

Length of peduncle.—3.3-4.2 mm.

Color of peduncle.—Moderate olive green (R.H.S. 146A and J.H.S. 3508).

Number of flowers.—Medium (approximately 9-14).

Flower bearing.—In a cluster (as illustrated).

Reproductive organs.—1 pistil and 4 stamens.

Pollen.—Brilliant greenish-yellow (R.H.S. 6C, J.H.S. 2704) in coloration.

Flower fragrance.—Absent.

Flowering time.—Early.

Flowering duration.—Long.

When planted during March, the plant commonly blossoms from early April to November. A bloom cluster is commonly present for approximately 2 to 3 weeks, and an individual bloom within the cluster commonly lasts for approximately 7 to 10 days on the plant.

Physiological and ecological characteristics: High resistance to diseases and pests, particularly powdery mildew. High tolerance to heat and drought, high tolerance to rain, and medium tolerance to cold.

This new variety of Verbena plant is most suitable for flower bedding and potting, particularly in planters. It is further excellent for use as ground cover.

I claim:

1. A new and distinct variety of Verbena plant having the FOLLOWING combination of characteristics:
(a) exhibiting a broad spreading growth habit with long stems,
(b) forming in abundance clusters of attractive deep purplish pink blossoms that remain on the plant for an extended period of time, and
(c) exhibiting good tolerance to rain, heat, drought, and diseases.
